(19) United States
(12) Patent Application Publication
(10) Pub. No.: US 2004/0209806 A1
(43) Pub. Date: Oct. 21, 2004

(54) Regulation of allergen induced gene

(75) Inventor: Marc Elliot Rothenberg, Cincinnati, OH (US)

Correspondence Address: WOOD, HERRON & EVANS, LLP, 2700 CAREW TOWER, 441 VINE STREET, CINCINNATI, OH, 45202, US

(73) Assignee: Children's Hospital Medical Center

(21) Appl. No.: 10/759,288

(22) Filed: Jan. 16, 2004

Related U.S. Application Data
(60) Provisional application No. 60/440,934, filed on Jan. 17, 2003.

(51) Int. Cl. A61K 48/00; A61K 38/17

(52) U.S. Cl. 514/12; 514/44

(57) ABSTRACT

A composition and method for alleviation of an allergic response by regulation of trefoil factor-2 (TFF2) expression. TFF2 is also disclosed as a marker for assessment of an allergic patient's status, for example, monitoring inflammation and/or tissue repair in a lung of an asthmatic patient. Regulation of TFF2 is involved in the pathogenesis of allergic lung inflammation and other allergen-induced conditions, for example, up-regulation of TFF2 may exert a protective effect by reducing acid secretion and/or increasing epithelial cell proliferation to promote healing.

### RELATED APPLICATION

This application claims priority to U.S. Provisional Patent Application Serial No. 60/440,934 filed Jan. 17, 2003, now pending and expressly incorporated by reference herein in its entirety.

The U.S. Government has a paid-up license in this invention and the right in limited circumstances to require the patent owner to license others on reasonable terms as provided for by the terms of Grant Nos. R01 A142242-05 and R01 A145898-03 awarded by the NIH.

### FIELD OF THE INVENTION

The invention relates to compositions and methods to regulate expression of trefoil family factor 2 peptide associated with an allergic response such as asthma.

### BACKGROUND

Asthma is a complex chronic inflammatory pulmonary disorder. Despite intense research, the incidence of asthma is on the rise and it is the chief diagnosis responsible for pediatric hospital admissions.

Asthma research has largely focused on analysis of the cellular and molecular pathways induced by allergen exposure in sensitized animals, including humans. Studies have identified elevated production of IgE, mucus hypersecretion, airway obstruction, inflammation and enhanced bronchial reactivity to spasmogens in the asthmatic response. Clinical and experimental investigations have demonstrated a strong correlation between the presence of CD4⁺ T helper 2 lymphocytes (Th2 cells) and disease severity, which suggested a role for these cells in the pathophysiology of asthma. Th2 cells are thought to induce asthma through the secretion of a variety of cytokines (IL-4, -5, -6, -9-10, -13, -25) which activate inflammatory and residential effector pathways both directly and indirectly. IL-4 and IL-13 are produced at elevated levels in the asthmatic lung and are thought to be key regulators of many of its hallmark features.

Attention has recently focused on the pathogenesis of airway remodeling in the setting of chronic airway inflammation. Mesenchymal cell signaling, induced by Th2 cytokines, has an active role in chronic injury and repair processes in response to allergen triggered inflammation. Thus, multiple therapeutic agents likely interfere with specific inflammatory pathways, and the development of the asthma phenotype is likely to be related to the complex interplay of a large number of additional genes, and their polymorphic variants.

Compositions and methods to alleviate asthma by such mechanisms are thus desirable.

### SUMMARY OF THE INVENTION

One embodiment of the invention is directed to a method to reduce an allergic response in a patient by regulating expression of trefoil factor-2 (TFF2). This may alleviate symptoms of asthma in an airway, lung, trachea, and/or lung fluid (bronchoalveolar lavage fluid), or alleviate allergic symptoms in skin, eyes, nose, and/or gut.

Another embodiment of the invention is a pharmaceutical composition containing an effector of TFF2 expression in a formulation and an amount sufficient to regulate the DNA encoding TFF2, the mRNA encoding TFF2, and/or the TFF2 protein produced. The effector may be an inhibitor of STAT6 and/or an inhibitor of a Th2 cytokine, such as interleukin (IL)-4 or IL-13. The inhibitors may be small molecule inhibitors, anti-sense inhibitors, and/or transcriptional inhibitors.

Another embodiment of the invention is a physiological assessment method whereby patient levels of TFF2 are determined, thereby providing an assessment of the patient's pulmonary status. TFF2 may be determined in lung fluid, lung biopsy specimens, sputum, mucus, nasal washings, and/or blood. The specimen is analyzed to determine TFF2 DNA, mRNA, and/or protein. As one example, Southern, Northern, or Western blots may be performed on biopsy specimens and treated with a probe to determine DNA, RNA, and protein, respectively. As another example, tissue may be appropriately stained and examined microscopically. An increased level of TFF2 would indicate an inflammatory process and/or a chronic repair process.

Another embodiment of the invention is a prophylactic or therapeutic method by providing TFF2 in a pharmaceutically acceptable composition to the lung. The method may reduce lung pH to treat lung inflammation, and/or may enhance epithelial cell repair in the lung to treat lung inflammation.

Another embodiment of the invention is a method to enhance repair of inflamed lung tissue by administering a TFF2 regulator in a pharmaceutically acceptable formulation and amount to up-regulate TFF2 expression. Enhanced TFF2 expression reduces acid secretion and/or enhances proliferation of epithelial cells, both of which promote repair of inflamed tissue.

Another embodiment of the invention is a treatment method for an allergic patient. The patient is administered an amount and formulation of a pharmaceutical composition containing at least one compound capable of differentially regulating an allergen-induced gene in a patient. The compound may affect STAT6 as an anti-sense compound, a small molecule inhibitor, or a transcription inhibitor.

These and other advantages will be apparent in light of the following figures and detailed description.

### BRIEF DESCRIPTION OF THE DRAWINGS

FIG. 1 illustrates expression of TFF2 by microarray analysis during induction of experimental asthma in mice challenged with the allergens Aspergillus fumigatus (ASP) (FIG. 1A), and ovalbumin (OVA) (FIGS. 1B and 1C).

FIG. 2 shows Northern blots and ethidium bromide stained RNA gels demonstrating TFF2 expression in mice challenged with ASP (FIG. 2A) and mice twice challenged with OVA (FOG. 2B). FIG. 2C shows TFF2 expression kinetics in mice challenged with OVA.

FIG. 3 shows Northern blots and ethidium bromide stained RNA gels demonstrating TFF2 expression in the presence and absence of STAT6 in IL4 transgenic (IL-4tg) and wild type (wt) mice (FIG. 3A), mice treated with IL4 (FIG. 3C), and mice treated with IL-13 (FIG. 3B).

FIG. 4 shows Northern blots and ethidium bromide stained RNA gels demonstrating TFF2 expression in the presence and absence of STAT6 in mice challenged with OVA (FIG. 4A) or ASP (FIG. 4B), and in IL-13 gene deleted mice (FIG. 4C).

### DETAILED DESCRIPTION

Trefoil peptides are small (7-12 kDa) protease-resistant proteins, composed of a characteristic three loop structure formed by three conserved cysteine disulfide bonds. They are secreted by the gastrointestinal mucosa in a lineage-specific manner. Trefoil factors are critically involved in responses to intestinal injury, primarily by their ability to promote epithelial restitution, the rapid spreading and migration of existing epithelial cells following injury.

The trefoil factor family peptide 2 (TFF2) is involved in repair responses associated with allergic lung disorders. TFF2, also known as spasmolytic polypeptide, is expressed in the stomach and to a lesser extent in the proximal duodenum and biliary tract. The other family members, TFF1 and TFF3, are expressed and secreted predominantly by gastric pit cells and intestinal goblet cells, respectively.

While TFF2 is expressed and secreted preferentially by gastric mucus neck cells, it is up-regulated in diverse pathologic conditions of the gastrointestinal tract including ulceration, inflammatory bowel disease, Helicobacter pylori infection, and by injury promoted by nonsteroidal anti-inflammatory drugs. In these conditions, TFF2 is thought to regulate acid production, stabilize the mucin gel layer by directly interacting with mucin proteins, and promote healing.

Expression and regulation of TFF2 in lung inflammation, such as occurs in allergy, asthma, etc., is disclosed. TFF2 was involved in the remodeling and repair responses associated with allergic lung disorders. Furthermore, because TFF2 directly interacted with mucin proteins, molecules that are over-produced in the asthmatic lung, their involvement in allergic lung responses was determined.

As demonstrated in transcript expression profiles, TFF2 was up-regulated in lung tissue from animals that were challenged with an allergen, either ovalbumin (OVA) or Aspergillus fumigatus (ASP), in experimentally-induced asthma. TFF2 was also specifically regulated by interleukin-4 (IL-4) and IL-13. In addition, STAT6 was required for TFF2 induction by OVA and by IL-13, but STAT6 was not required for TFF2 induction by ASP or by IL-4.

Animals (wild type BALB/c and STAT6-deficient BALB/c mice) were administered intraperitoneal (i.p.) injections of OVA, then were administered intranasal ASP antigen. Alternatively, animals were administered IL-13.

Genes were analyzed which were not specific to a particular experimental regimen, thus, two independent models of asthma were used. The allergen-induced genes which overlapped in these two independent models were analyzed using global transcript profile analysis. Both asthma models, however, have similar phenotypes, including Th2 associated eosinophilic inflammation, mucus production, and airway hyperresponsiveness (AHR).

In one model, mice were sensitized by i.p. injections of the allergen OVA in the presence of the adjuvant alum on two occasions separated by fourteen days. Subsequently, mice were challenged with intranasal OVA or control saline on two occasions separated by three days. Eighteen hours after the last allergen challenge, the lung was harvested for RNA analysis. In another model, experimental asthma was induced by the Aspergillus fumigatus antigen, a ubiquitous and common aeroallergen. This model involved a unique mucosal sensitization route (intranasal), compared with the OVA model. Lung RNA was obtained eighteen hours after nine doses of intranasal Aspergillus fumigatus allergen or saline challenges.

Analysis of microarray data indicated increased expression of TFF2, but not TFF1 or TFF3, during asthma induced by either of OVA or ASP. Northern blot analysis revealed that TFF2 was not expressed in the lung under normal conditions, but its expression was markedly induced by allergen challenge. This TTF2 up-regulation depended upon STAT6 in the OVA-challenged mice, but not in the ASP-challenged mice. Additionally, TTF2 was up-regulated in IL-13 challenged mice by a pathway which depended upon the protein STAT6, and also by a pathway which was independent of the protein STAT6.

Whole lung RNA was analyzed by DNA microarray hybridization. RNA was extracted using the Trizol (Invitrogen, Carlsbad Calif.) reagent according to manufacturer's instructions. Following Trizol purification, RNA was repurified with phenol-chloroform extraction and ethanol precipitation.

Microarray hybridization was performed by the Affymetrix Gene Chip Core facility at Cincinnati Children's Hospital Medical Center. Briefly, RNA quality was first assessed using the Agilent bioanalyzer (Agilent Technologies, Palo Alto Calif.) and only those samples with 28S/18S ratios between 1.3 and 2 were subsequently used. RNA was converted to cDNA with Superscript choice for cDNA synthesis (Invitrogen, Carlsbad Calif.) and subsequently converted to biotinylated cRNA with Enzo High Yield RNA Transcript labeling kit (Enzo Diagnostics, Farmingdale N.Y.). After hybridization to the murine U74Av2 GeneChip (Affymetrix, Santa Clara Calif.), the gene chips were automatically washed and stained with streptavidin-phycoerythrin using a Fluidics System. The chips were scanned with a Hewlett Packard GeneArray Scanner. This analysis was performed with one mouse per chip (n≧3 for each allergen challenge condition and n≧2 for each saline challenge condition).

For Northern blot analysis, RNA was extracted from the lungs of wild-type Balb/c mice, IL-4 Clara cell 10 lung transgenic mice as described by Rankin et al., Proc. Natl. Acad. Sci USA 93:7821-5 (1996), which is expressly incorporated by reference herein in its entirety. The mice contained wild-type or deleted copies of the gene for STAT6. RNA was also extracted from the lungs of mice treated with saline or recombinant murine IL-13, as described by Pope et al., J. Allergy Clin. Immunol. 108:594-601 (2001), and by Zimmermann et al., J. Immunol. 165:5839-46 (2000), each of which is expressly incorporated by reference herein in its entirety. Hybridization was performed with ³²P-labeled cDNA encoding the sequence-confirmed murine TFF2 (I.M.A.G.E. 438574) or TFF3 (I.M.A.G.E. 1166710), obtained from American Type Culture Collection, Rockville Md..

From data image files, gene transcript levels were determined using algorithms in the Microarray Analysis Suite Version 4 software (Affymetrix). Global scaling was performed to compare genes from chip to chip; thus, each chip was normalized to an arbitrary value (1500). Each gene is typically represented by a probe set of 16 to 20 probe pairs. Each probe pair consists of a perfect match oligonucleotide and a mismatch oligonucleotide that contains a one base mismatch at a central position. Two measures of gene expression were used: absolute call and average difference. Absolute call is a qualitative measure in which each gene is assigned a call of present, marginal, or absent based on hybridization of the RNA to the probe set. Average difference is a quantitative measure of the level of gene expression, calculated by taking the difference between mismatch and perfect match of every probe pair and averaging the differences over the entire probe set.

Differences between saline and allergen-treated mice were also determined using the GeneSpring software (Silicon Genetics, Redwood City Calif.). Data were normalized to the average of the saline-treated mice. Gene lists were created which contained genes with p<0.05 and >2-fold change (using genes that received a present call based on the hybridization signal).

Balb/c mice were obtained from the National Cancer Institute (Frederick Md.) and STAT6-deficient mice (Balb/c) were obtained from Jackson Laboratory (Bar Harbor Me.). All mice were housed under specific pathogen-free conditions.

Asthma models were induced as described by Mishra et al., J. Biol. Chem. 276:8453 (2001), which is expressly incorporated by reference herein in its entirety. Briefly, ovalbumin-induced asthma was induced by i.p. injections of OVA and 1 mg aluminum hydroxide (alum) separated by two weeks, followed by two doses of intranasal (i.n.) OVA or saline challenge two weeks later. Aspergillus fumigatus antigen-induced asthma was induced over the course of three weeks by repeated intranasal inoculation of antigen.

RNA obtained from the saline- and allergen-challenged mice was subjected to microarray analysis utilizing the Affymetrix chip U74Av2 which contains oligonucleotide probe sets representing 12,423 genetic elements, one of the largest collection of characterized mouse genes commercially available. Allergen-challenged mice (OVA or Aspergillus) were compared to their respective saline control mice (n=3-6 mice in each experimental group) and genes which showed at least a two-fold statistically significant increase (p<0.05) following allergen challenge were identified.

Compared with saline-challenged mice, OVA-challenged mice had 496 genes induced and Aspergillus fumigatus-challenged mice had 527 genes induced. The majority (59% of OVA and 55% of Aspergillus) of the induced transcripts overlapped between the two experimental asthma models.

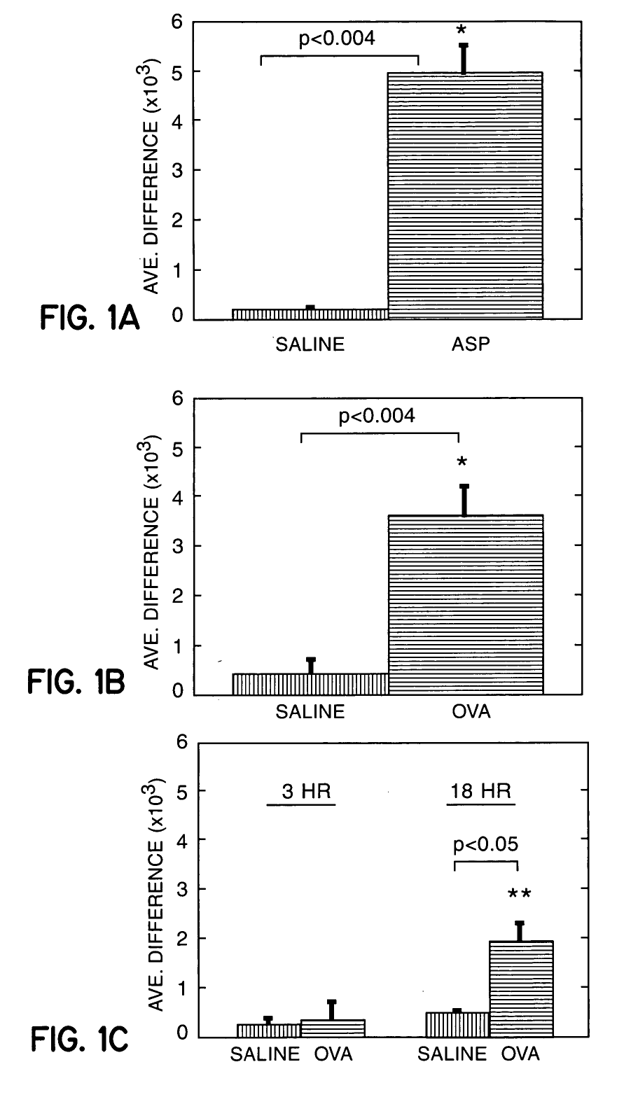

DNA microarray analysis identified TFF2 as an allergen-induced gene in experimental asthma. FIGS. 1A-C illustrate expression of TFF2 by microarray analysis during induction of experimental asthma. FIG. 1A shows expression of TFF2 in mice challenged with Aspergillus fumigatus (ASP). FIGS. 1B and 1C show expression of TFF2 in mice challenged with ovalbumin (OVA). Data were from quantitative microarray analysis, with the average difference for the hybridization signal following saline and allergen challenge depicted. Values represent the mean, and error bars represent the standard deviation. Statistical significance is indicated.

A set of 291 genes that were commonly involved in disease pathogenesis, rather than unique to a particular allergen or mode of disease induction, were identified. These “asthma signature” genes enabled definition of new pathways involved in the pathogenesis of allergic airway inflammation to be elucidated, including a high level of transcripts for TFF2 in the asthmatic lung.

The results of a kinetic analysis after the first OVA challenge are shown in FIG. 1C. TFF2 was detectable 18 hours after the first allergen challenge, but not at three hours. Microarray analysis revealed very specific dysregulation of TFF2 compared with other TFFs. For example, the hybridization signals for TFF1 was below background in the saline- and allergen-challenged lung and, while the TFF3 mRNA signal was present, it remained unchanged in response to allergen challenge (data not shown).

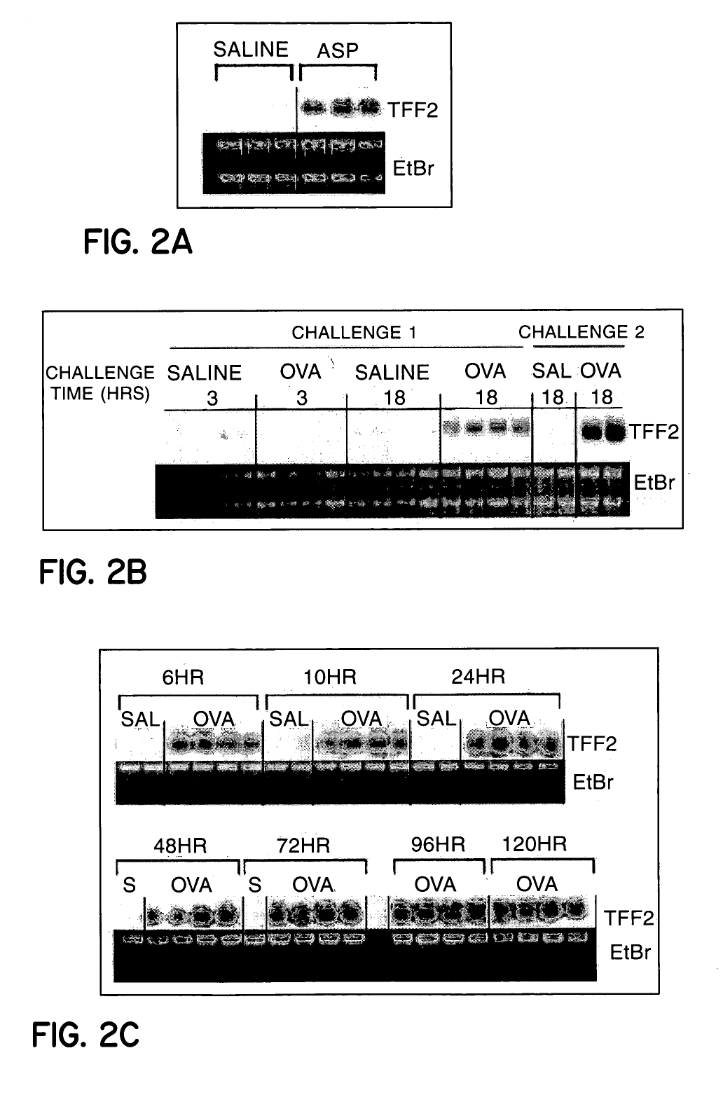

FIGS. 2A-C show Northern blots and ethidium bromide stained gels demonstrating TFF2 expression following allergen challenge. Aspergillus fumigatus-challenged mice had marked expression of TFF2, compared with mice challenged with saline. FIG. 2A demonstrates expression of TFF2 following i.n. administration of Aspergillus fumigatus, with an autoradiograph exposure time of 72 hours. Additionally, there was a time- and dose-dependent induction of TFF2 during the progression of OVA-induced experimental asthma. FIG. 2B demonstrates expression of TFF2 following OVA challenge. Time points include 3 and 18 hours after one allergen challenge and 18 hours after two challenges. TFF2 was induced 18 hours after the first allergen challenge and to an even greater extent following two allergen challenges. As shown in FIG. 2C, subsequent kinetic analysis revealed that TFF2 expression was maximal by 10 hours after the second challenge, and this level was maintained through 120 hours. TFF3 mRNA was not detectable by Northern blot analysis of the same experimental asthma lung samples, although they were detected in a Northern blot prepared from gastrointestinal tissue RNA (data not shown).

Because asthma is a Th2-associated process, it was determined whether overexpression of IL-4, particularly in the lungs, was sufficient for induction of TFF2. Mice that overexpress the IL-4 transgene in pulmonary epithelium (under the control of the Clara cell 10 promoter) have several features of asthma, including eosinophil-rich inflammatory cell infiltrates, mucus production, and changes in baseline airway tone.

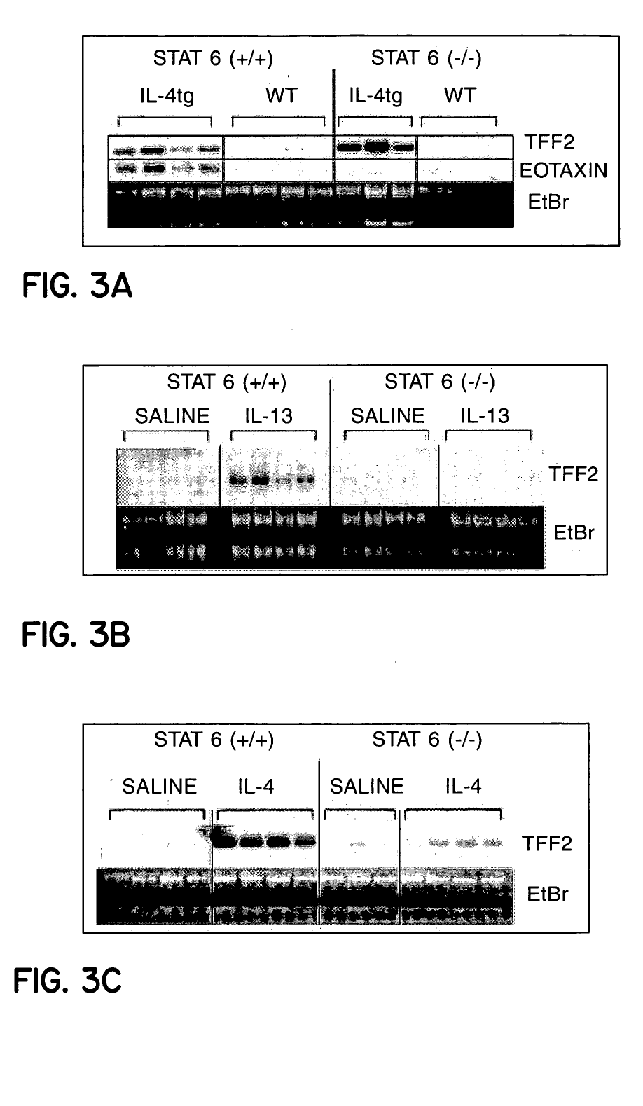

Overexpression of IL-4 potently induced lung TFF2 in vivo. FIGS. 3A-C show Northern blots and ethidium bromide stained RNA gels demonstrating regulation of TFF2 by interleukins (IL)-4 and -13, and by STAT6. Each lane represent a separate animal.

FIG. 3A demonstrates TFF2 mRNA expression in IL-4 lung transgenic (Tg) or wild-type (WT) mice carrying wild-type (+/+) or gene deleted (−/−) copies of STAT6. As shown in the Figure, TFF2 mRNA was induced by the IL-4 transgene.

IL-4 and IL-13 induction of lung TFF2 was differentially dependent on STAT6. IL-4 and IL-13 share similar signaling requirements, such as utilization of the IL-4Rα chain and the induction of janus kinase 1 and STAT6. A subset of their responses has been shown to be STAT6 dependent.

To determine the role of STAT6 in the induction of TFF2 in vivo, the lungs of IL-4 transgenic mice that contained wild-type-or gene targeted deletion of STAT6 were examined. These mice were generated by mating IL-4 lung transgenic mice with STAT6-deficient mice, as described by Zimmermann et al., J. Immunol. 165:5839-46 (2000), which is expressly incorporated by reference herein in its entirety. The results are also shown in FIG. 3A.

IL-4-induced TFF2 mRNA expression was not abrogated by the loss of STAT6, although other IL-4-induced lung genes have been reported to be STAT6 dependent (Zimmermann et al., J. Immunol. 165:5839-46 (2000)). For verification, expression of eotaxin-I in these mice was evaluated. As shown in FIG. 3A, IL-4-induced eotaxin mRNA expression was completely dependent upon STAT6. FIG. 3C demonstrates TFF2 nRNA expression when IL-4 or saline was delivered to wild type (+/+) or STAT6 deficient (−/−) mice.

FIG. 3B demonstrates TFF2 mRNA expression with IL-13 or saline administration to wild-type (+/+) or STAT6-deficient (−/−) mice. IL-13 is a cytokine involved in the development of several features of experimental asthma, including eosinophilic inflammation, chemokine induction, mucus production, and AHR. To determining if lung TFF2 was also induced by IL-13, repeated intranasal applications of IL-13 were administered to anesthetized mice. As shown in FIG. 3B, IL-13 administration induced marked levels of lung TFF2 mRNA compared with saline treated control mice. The dependence of STAT6 on the ability of IL-13 to induce TFF2 was evaluated. IL-13 was administered to wild-type and STAT6-deficient mice. As shown in FIG. 3B, IL-13 failed to induce TFF2 in the absence of STAT6.

Collectively, these results demonstrated that TFF2 induction by IL-13, but not by the IL-4 transgene, occurred by a STAT6-dependent mechanism.

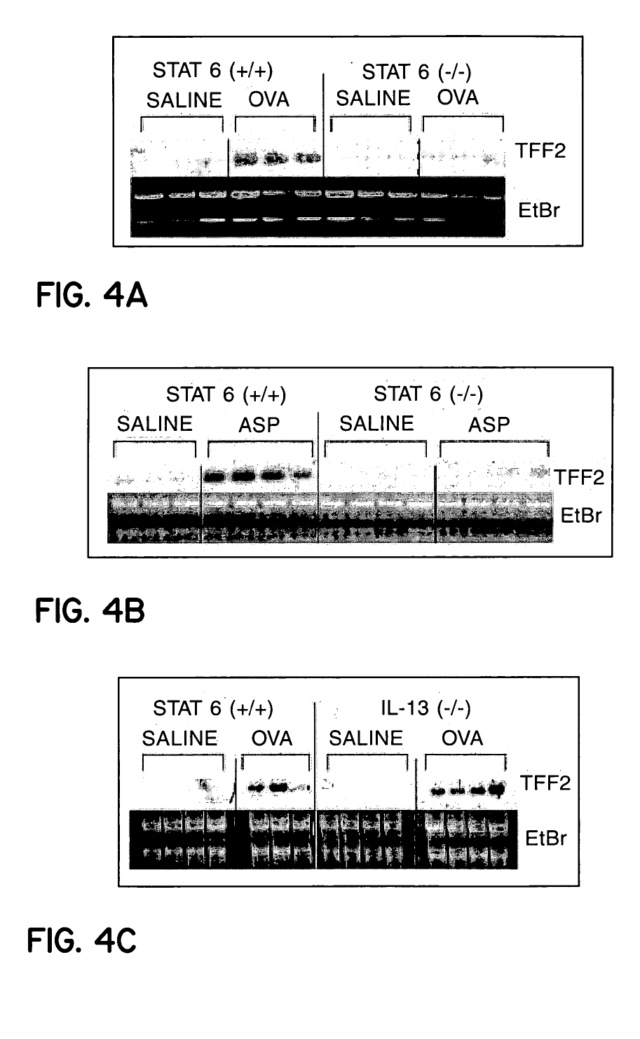

Allergen-induced TFF2 expression was differentially regulated by STAT6. FIGS. 4A-B show Northern blots and ethidium bromide stained RNA gels demonstrating STAT6-dependent regulation of TTF2 induced by either OVA (FIG. 4A, 4C) or Aspergillus fumigatus (FIG. 4B). Experimental asthma was induced in wild-type (+/+) or STAT6 gene deleted (−/−) mice.

The dependence of STAT6 on allergen-induced TFF2 expression would help determine if allergen-induced TFF2 was predominantly downstream from IL-13 signaling. As shown in FIG. 4A, mice deficient in STAT6 showed reduced lung TFF2 following OVA challenge; as a control, wild-type mice displayed readily detectable lung TFF2. In contrast, when the STAT6 requirement was examined in the Aspergillus fumigatus-induced model of experimental asthma, there was strong induction of TFF2 even in the absence of STAT6. For example, and with reference to FIG. 4B, the levels of TFF2 mRNA were comparable in the wild-type and STAT6 mice following Aspergillus fumigatus treatment.

OVA-induced experimental asthma in IL-13 gene-targeted mice was also evaluated. As shown in FIG. 4C, IL-13 gene-targeted mice had reduced OVA-induced TFF2 expression. OVA-induced TFF2 occurred downstream from IL-13 and STAT6 signaling.

These results demonstrated that the mechanism of allergen-induced TFF2 induction varied with distinct experimental regimes. The OVA-induced model was regulated by a Th2-associated STAT6 pathway. The Aspergillus fumigatus model induced TFF2 by a pathway that was primarily independent of STAT6.

To understand the complex mechanisms involved in the pathogenesis of asthma, transcript expression profile analysis was used to define a set of “asthma signature” genes. The discovery of TFF2 as an asthma-associated gene indicated this molecule had properties potentially important in asthmatic responses. TFF2 was not previously implicated in the pathogenesis of asthma.

Allergic lung inflammation, triggered by diverse allergens and modes of disease induction, was associated with marked and specific ectopic expression of TFF2, but not TFF1 or TFF3, in the lung. This is in contrast to prior work which found that expression of TFFs, particularly TFF2, was primarily restricted to the gastrointestinal tract.

The Th2 cytokines IL-4 and IL-13 were potent inducers of TFF2 in the lung. Thus, allergen-induced TFF2 was mediated, at least in part, by IL-4 and IL-13. IL-4 and IL-13 are related cytokines that share a similar signaling mechanism (e.g. utilization of a common receptor subunit (IL-4Rα chain) and activation of STAT6). Both of these cytokines were known to play roles in asthma, but the mechanisms by which they induced various elements of the asthmatic response (e.g. AHR, mucus production, and airway remodeling) were only partially understood. The present invention shows that the pathogenesis of IL-4/IL-13-associated allergic lung responses is mediated by TFF2, at least in part. Injury-associated epithelial hyperplasia and epithelial differentiation (e.g. mucus cell metaplasia), processes known to be regulated by TFF2 in the gastrointestinal tract, may also be mediated by TFF2 in the lung. TFF2 also inhibited mucus production.

TFF2 was induced by both IL-4 and IL-13, but STAT6 was not a requisite for TFF2 induction. For example, Aspergillus fumigatus- and IL-4-induction of TFF2 occurred at comparable levels in STAT6-deficient and wild-type mice. However, in contrast, IL-13 and OVA-induced TFF2 were attenuated in STAT6-deficient mice. These data are consistent with studies that have shown distinct and overlapping mechanisms for the involvement of IL-4 and IL-13 in experimental asthma (Wills-Karp, M., J. Allergy Clin. Immunol. 107:9-18 (2001)). Additionally, while OVA and Aspergillus both induce experimental asthma, Aspergillus was capable of inducing Th2 responses independent of adjuvant. This indicated that both allergens employ distinct mechanisms for asthma induction.

Th2 cytokine mediated TFF2 induction is likely to occur by an indirect mechanism. Consistent with an indirect mechanism, the TFF2 promoter is not known to contain a STAT binding site. GATA6, a transcription factor normally expressed in the heart and gastrointestinal tract, is used for TFF2 induction and may have a role in TFF2 expression in the lung.

Under healthy conditions, TFF2 is predominantly expressed in the stomach with lower levels in the proximal duodenum and biliary tract. In the stomach, TFF2 is expressed by gastric mucus neck cells, and is secreted onto the mucosal surface associated with mucin proteins. TFF2 is up-regulated in diverse injury-associated pathological conditions in the gastrointestinal tract, including ulceration associated with Helicobacter pylori infection, nonsteroidal anti-inflammatory drug use, and Crohn's disease. In all of these states, TFF2 expression appeared to be related to the proliferative zone of the mucosa, suggesting that TFF2 may be involved in regulating epithelial proliferation in response to injury. The asthmatic lung is characterized by a large increase in epithelial proliferation.

TFF2 has been linked with inhibiting acid production in the stomach. The asthmatic airway is characterized by an acidified environment that appears to be responsible for the oxidation of nitrite to nitric oxide, a process that strongly correlates with airway inflammation. There is a role for TFF2 in promoting mucosal healing through inhibition of acid secretion and stimulation of epithelial proliferation. Allergen-induced TFF2 may play a role in regulating several features associated with the pathogenesis of asthma, including acidification of the airway and epithelial proliferation. These results raise the importance of subjecting TFF2-deficient mice to the induction of experimental asthma.

TFF2 is an allergen-induced gene in the asthmatic lung. The Th2 cytokines IL-4 and IL-13 induced TFF2. TFF2 induction occurred by STAT6 dependent (as in the case of IL-13 and OVA) and independent (as in the case of IL-4 and Aspergillus fumigatus) mechanisms. Thus, TFF2 was involved with the pathogenesis of asthma. TFF2 involvement included processes known to be regulated by TFF2 in the gastrointestinal tract, including epithelial proliferation and acid production. The allergic lung responses shared pathogenic mechanisms with disease processes in the gastrointestinal tract.

Compositions may be pharmaceutically acceptable formulations of TFF2 or compounds that effect the expression of trefoil peptides such as TFF2. Their concentration in the composition may be prepared for doses ranging from about 0.01 mg/kg to about 100 mg/kg of body weight. The amounts of compound in the composition may vary depending on the type of formulation.

Compositions affecting TFF2 may be small molecule inhibitors, anti-sense inhibitors, and/or transcriptional inhibitors of STAT6 or Th2 cytokine inhibitors. Compositions may be administered to a mammal, such as a human, either prophylactically or in response to a specific condition or disease. For example, the composition may be administered to a patient with asthmatic symptoms and/or allergic symptoms. The composition may be administered non-systemically such as by inhalation, aerosol, drops, etc.; systemically by an enteral or parenteral route, including but not limited to intravenous injection, subcutaneous injection, intramuscular injection, intraperitoneal injection, oral administration in a solid or liquid form (tablets (chewable, dissolvable, etc.), capsules (hard or soft gel), pills, syrups, elixirs, emulsions, suspensions, etc.). As known to one skilled in the art, the composition may contain excipients, including but not limited to pharmaceutically acceptable buffers, emulsifiers, surfactants, electrolytes such as sodium chloride; enteral formulations may contain thixotropic agents, flavoring agents, and other ingredients for enhancing organoleptic qualities.

Different routes of administration may be used. As examples, an intravenous administration may be continuous or non-continuous; injections may be administered at convenient intervals such as daily, weekly, monthly, etc.; enteral formulations may be administered once a day, twice a day, etc. Instructions for administration may be according to a defined dosing schedule, or an “as needed” basis.

Different body parts may be affected by allergens. Thus, evaluation of TTF2 levels, and regulation of TTF2 expression, may occur in various organs. As one example, in asthma, the airway, lung, trachea, respiratory tract tissue, respiratory fluid, throat, mucus, nasal washings, and/or lung fluid (bronchoalveolar lavage fluid) would be targeted. As another example, allergic symptoms could manifest in the skin (hives, rash, urticaria), eyes (inflammation), nose (rhinitis), and/or gut.

The diagnostic ability of TFF2 is also disclosed. Qualitative and quantitative determinations of TFF2 are markers of an inflammatory process. Thus, TFF2 determination may be used to assess a patent's clinical status, phenotype, genotype, drug response, and/or prognosis and determine single nucleotide polymorphisms. An increased level of TFF2 in pulmonary tissue obtained from a biopsy site would indicate an inflammatory process and/or a chronic repair process. TFF2 may be determined in lung fluid, lung biopsy specimens, sputum, mucus, nasal washings, and/or blood. The specimen is analyzed so that TFF2 DNA, mRNA, and/or protein is determined. As one example, Southern, Northern, or Western blots may be performed on biopsy specimens and treated with a probe to determine DNA, RNA, and protein, respectively. As another example, the tissue may be histologically evaluated, for example, by appropriate staining and microscopic examination. Such methods are known to one skilled in the art.

TFF2 provided to the lung may reduce lung pH to treat lung inflammation, and/or may enhance epithelial repair in the lung to treat lung inflammation.

Other variations or embodiments of the invention will also be apparent to one of ordinary skill in the art from the above description including those described in Am. J. Respir. Cell. Mol. Biol. 29:458, (2003), which is expressly incorporated by reference herein in its entirety. Thus, the forgoing embodiments are not to be construed as limiting the scope of this invention.

What is claimed is:

1. A method to mitigate an allergic response in a patient comprising enhancing expression of trefoil factor-2 (TFF2) to thereby mitigate the allergic response in the patient.

2. The method of claim 1 wherein the mitigation encompasses altered mucus production, promoted cell hyperplasia, and combinations thereof.

3. The method of claim 1 wherein expression is enhanced in an airway, lung, trachea, respiratory tract, or bronchoalveolar lavage fluid.

4. The method of claim 1 wherein expression is enhanced in a body part affected by an allergy.

5. The method of claim 4 wherein the body part is selected from the group consisting of skin, eye, nose, throat, gut, and combinations thereof.

6. The method of claim 1 wherein a pharmaceutical composition of a regulatory compound is administered systemically.

7. The method of claim 1 wherein administration is by a route selected from the group consisting of intravenously, intranasally, intratracheally, subcutaneously, intramuscularly, orally, intraperitoneally, and combinations thereof.

8. The method of claim 1 wherein expression is enhanced by at least one of interleukin-4 (IL-4) or interleukin-13 (IL-13).

9. The method of claim 1 wherein the allergic response is selected from the group consisting of allergic rhinitis, asthma, eczema, and combinations thereof.

10. The method of claim 8 further comprising II-13 and signal transducer and activator of transcription (STAT) 6.

11. A pharmaceutical composition comprising an effector of trefoil factor-2 (TFF2) expression in a pharmaceutically acceptable formulation and amount sufficient to enhance an amount of DNA encoding TFF2, mRNA encoding TFF2, TFF2 protein, or combinations thereof.

12. The composition of claim 11 comprising an activator of STAT6, an activator of a Th2 cytokine, or combinations thereof.

13. The composition of claim 12 comprising activators selected from the group consisting of small molecule activators, oligonucleotide activators, transcriptional activators, and combinations thereof.

14. The composition of claim 11 in a formulation for administration to an allergic patient.

15. The composition of claim 11 in a formulation for administration to an asthmatic patient.

16. A physiological lung assessment method comprising determining a level of trefoil factor-2 (TFF2) in a patient to assess a patient condition selected from the group consisting of clinical status, phenotype, genotype, drug response, prognosis, determine single nucleotide polymorphisms, and combinations thereof.

17. The method of claim 16 wherein TFF2 is determined in lung fluid, lung biopsy, sputum, mucus, nasal washings, respiratory tract tissue, respiratory tract fluid, blood, and combinations thereof.

18. The method of claim 16 wherein TFF2 DNA, mRNA, protein, or combinations thereof are determined.

19. The method of claim 16 wherein an increased level of TFF2 indicates an inflammatory process.

20. The method of claim 16 wherein an increased level of TFF2 indicates a chronic repair process.

21. The method of claim 16 wherein the patient is at least one of allergic or asthmatic.

22. A prophylactic or therapeutic method for a patient comprising providing trefoil factor-2 (TFF2) in a pharmaceutically acceptable composition to a lung of a patient in an amount sufficient to cause at least one of reduced lung acidity or enhanced lung epithelial cell repair, thereby treating lung inflammation.

23. A treatment method comprising providing to an allergic patient an amount and formulation of a pharmaceutical composition containing at least one compound capable of differentially regulating an allergen-induced gene in a patient.

24. The method of claim 23 wherein the allergen-induced gene encodes trefoil factor-2.

25. A method to enhance repair of allergy-induced inflamed tissue comprising administering to a patient a composition comprising a regulator of trefoil factor-2 (TFF2) expression in a pharmaceutically acceptable formulation and in an amount sufficient to up-regulate TFF2 expression to result in at least one of reduced acid secretion or enhanced epithelial cell proliferation for enhanced repair of the inflamed tissue.

26. The method of claim 25 wherein the regulator of TFF2 expression is a Th2 cytokine.

27. The method of claim 25 wherein the regulator of TFF2 expression is at least one of IL-4 or IL-13.

28. The method of claim 25 wherein the regulator of TFF2 expression further comprises at least one of transcription factor STAT6 or transcription factor GATA6.

29. The method of claim 28 wherein the regulator is at least one of a small molecule activator of STAT6, a STAT6 oligonucleotide, or an activator of STAT6 transcription.

30. The method of claim 25 wherein the inflamed tissue is at least one of airway, lung, trachea, bronchoalveolar lavage fluid, skin, eyes, throat, or nose.

31. The method of claim 25 wherein the patient is allergic or asthmatic.
